(19) United States
(12) Patent Application Publication
(10) Pub. No.: US 2003/0129210 A1
(43) Pub. Date: Jul. 10, 2003

(54) Uses of flaked cationic potato starch

(75) Inventor: Manjit Chowdhary, Princeton Junction, NJ (US)

Correspondence Address: VINSON & ELKINS L.L.P., 1001 FANNIN STREET, 2300 FIRST CITY TOWER, HOUSTON, TX, 77002-6760, US

(73) Assignee: Economy Mud Products Company, Houston, TX

(21) Appl. No.: 10/042,409

(22) Filed: Jan. 9, 2002

(51) Int. Cl. A61K 7/00; A61K 31/718

(52) U.S. Cl. 424/401; 514/60

(57) ABSTRACT

A cationic potato starch in solid flake form with a substitution value of about at least 0.01, where the starch has been chemically modified to be cationic. The starch has been demonstrated to have superior properties for use in such applications as personal care and agricultural drift control, or as an adhesive, a flocculent or a coagulant. The superior properties of the starch include superior film-forming, adhesive and crosslinking capabilities. The adhesive properties of the starch offer advantages over other starches in such service, or non-starch substitutes such as guar.

### TECHNICAL FIELD OF THE INVENTION

This invention relates generally to new uses of cationic potato starch in flake form, and more specifically to uses in the personal care and agriculture industries, and to uses of the starch as an adhesive, a flocculent or a coagulant.

### BACKGROUND OF THE INVENTION

Starch is known to be available from tubers such as potatoes. Chemically modified starch from potatoes is known in the art to be used in the manufacture of paper. U.S. Pat. No. 5,387,675 issued to Yeh discloses in its background section that such polymers have been proposed for such use.

Starch from other sources other than potatoes (e.g., from maize or other cereals) is known in the art to be used in personal care products such as hair spray (see USPTO publication 2001/0007655 A1), conditioner, and other hair styling products. Starch from sources other than potatoes is also known to be used as an adhesive (see U.S. Pat. Nos. 5,085,228; 5,454,862; and 5,855,659). Non-starch products such a guar are also conventionally used for some of these services.

The prior art indicates that hitherto, non-potato starch has been modified before use in personal care products, or as an adhesive. Techniques such as those disclosed, for example, in U.S. Pat. Nos. 5,387,675; 5,473,059; 5,609,711; 5,972,091; 5,403,871; and 6,207,176 involve the modification of a non-potato starch to a chemically- or physically-modified starch designed for a particular use. Such modifications include pre-gelatinization, acid hydrolysis, and mixing with plant germ.

Alternatively, guar compounds are known in the art to be useful for such service. See, for example, U.S. Pat. Nos. 5,550,224; 5,824,797; 5,874,096; and 6,210,689.

Conventional modified starches have been observed to have disadvantages when used in applications such as personal care and adhesives. In personal care applications, such modified starches have been rejected in favor of, for example, guar products, because the starch products have tended to have inferior film-forming properties when compared to the guar products. The starch products have further tended to have inferior cold water solubility, substantivity and lower viscosity than their guar gum product counterparts.

In some cases, guar is used as an alternative to starch in applications such as personal care, adhesives and drift control agents. Guar is recognized as a less than optimal substitute, however, because it lacks commercially useful adhesive properties.

In adhesives, water-soluble corn-based starches are known in the art for use as paper adhesives in, for example, the manufacture of cigarettes. It would be advantageous to use a starch with superior film-forming and adhesive properties in this and other adhesive applications.

In agricultural drift control, for example in crop spraying, guar is known and used conventionally as a drift agent. However, as noted, guar has poor or no adhesive properties. Alternative drift control agents with superior adhesive properties would enable the drift control agent to adhere the pesticide to the crop, and superior film-forming properties would enable the pesticide to be distributed evenly on the surface of the crop for improved pesticide performance. Superior film-forming properties would also be helpful to reduce evaporation from the plant. As a result, less pesticide would be needed on the plant to be effective. Additionally, there would be less runoff of pesticide into soil and waterways.

It will be further appreciated that industrial applications such as drilling mud preparation, wastewater treatment and environmental clean up always have need of an effective flocculent or coagulant. Superior film-forming properties in a cationic starch acting as such a flocculent or coagulant would bring advantage to these applications by facilitating removal of impurities. For example, large quantities of wastewater from construction site rainwater runoff often need to be treated before they can be discharged into municipal sewer systems. A water-soluble cationic starch added to such wastewater would attract and collect all kinds of “anionic trash,” facilitating its removal. Superior film-forming properties in the starch would further encourage rapid and even dispersal throughout the wastewater in attracting such impurities at an ionic level.

### SUMMARY OF THE INVENTION

These and other uses and applications as suggested in the background section are addressed by a cationic potato starch in solid flake form, with a substitution value of about at least 0.01. The flaked starch is obtained from potatoes using conventional methods. The flaked potato starch is then chemically modified to be cationic, again using conventional methods such as mixing with a cationic reagent.

It will be understood that henceforth, unless specified to the contrary, whenever this disclosure refers to cationic potato starch intended for use in the new applications described herein, this disclosure is referring to cationic potato starch in flake form.

Cationic potato starch in flake form has been demonstrated to have excellent film-forming and adhesive properties. In gel form, when used in personal care products, this starch is reported to have a very appealing feel to the skin. This favorable feel is most likely attributable to the superior film-forming properties, and to the cationic charge of the starch. The cationic charge allows products containing cationic potato starch to reside more effectively on the surface of the hair or skin on which the starch is applied, without being rinsed away. In addition, the superior adhesive properties of this starch in gel form allow personal products in which it is used, such as hair styling gel, hair conditioner or pore cleansing strips, to be more effective. Use of cationic potato starch allows such products to adhere better to the hair or skin on which they are used.

Cationic potato starch in flake form also exhibits excellent crosslinking properties, especially when crosslinked with crosslinking agents such as Borate, Zirconium or Titanium crosslinker. The covalent cationic structure of this starch allows strong bonds to be formed at the molecular level. It should be noted that cationic potato starch is water-soluble. The superior adhesive properties are evident inasmuch that this starch has been demonstrated to be very sticky when wet, and very non-sticky as soon as it is dry. Re-wetting can reclaim stickiness. This solubility in water facilitates this starch's preparation, delivery and curing. In subjective testing, cationic potato starch in flake form has been further demonstrated not to affect the color of surface texture of the substrates (such as hair, skin, paper or crops) to which it may be applied.

Cationic potato starch also exhibits excellent delivery and curing properties. Unlike conventional starches used in these applications, cationic potato starch does not have to be pre-cooked or pregelatinized. This starch forms a water-soluble gel that has simply to be mixed with water to form the gel. The superior film-forming properties of the gel encourage this starch to disperse quickly and evenly in solution at the ionic level. Once applied, the gel air-dries, with or without heat.

Accordingly, a technical advantage of the present invention is that exhibits superior film forming and adhesive properties. These superior properties allow cationic potato starch to be used advantageously over conventional starches or guars in applications such as personal care or drift control in agriculture, or as an adhesive, a flocculent or a coagulant.

A further technical advantage of the present invention is its superior ability to be crosslinked. This advantage is of particular benefit in personal care product applications, where, for example, styling or cleansing products may be caused to adhere better to substrates such as hair or skin for optimal performance.

A further advantage of the present invention is that cationic potato starch forms, at the ionic level, strong covalent bonds with anions in materials in which this starch is used as a flocculent or coagulant. These strong bonds encourage effective removal of anionic impurities from such materials.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION OF THE INVENTION

The cationic potato starch of the present invention is in solid flake form with a substitution value of about at least 0.01. This starch is derived from potatoes using conventional techniques well known in the art. These techniques include mixing with a cationic reagent, again according to methods well known in the art, to give the starch cationic properties. The cationic potato starch of the present invention is water-soluble. When added to water, this starch forms a gel having excellent film-forming, adhesive, delivery and curing properties. Covalent bonds at the ionic level further enable this starch to act as an effective flocculent or coagulant for removing impurities with negative charge. The ability of this starch to disperse quickly and evenly in solution further assists the starch in acting as an effective flocculent or coagulant.

Identifiable similarities with other starches notwithstanding, it will be nonetheless understood that cationic potato starch in flake form differs from other starches at the molecular level, recognizable by the way in which it crosslinks favorably with crosslinking agents such as zirconium, titanium, aluminum and borate-based crosslinking agents. It will be appreciated that these favorable crosslinking properties may be attributed to the chemical and/or physical changes that occur during flaking. It is believed that flake form assists crosslinking properties by preserving short branches better at the molecular level during preparation.

Advantageously, the cationic potato starch for the new uses described herein is used either in flake form, or in powder form having first been prepared in flake form. It has been noted, however, that the cationic potato starch used directly in flake form crosslinks even better than the powdered starch prepared originally in flake form.

An example of the cationic potato starch in flake form described in this disclosure is commercially available from Emsland Starch Gmbh as product “Empresol N.”

Cationic potato starch in flake form thus has advantageous uses in the personal care industry. As described in more detail above in the summary section, the superior film-forming and adhesive properties of cationic potato starch in gel form makes this starch an advantageous ingredient in personal care products.

As noted above, cationic potato starch in flake form has excellent crosslinking potential. This potential is believed to be due to cationic potato starch having less branching at the molecular level than, for example, cornstarch.

Borate crosslinking agents, for example, when used in personal care products with cationic potato starch in flake form, will enable the crosslinking to be reversible. Thus, the “setting” of the cationic potato starch in the product may be designed to be temporary to some degree. On the other hand, crosslinking agents such as Titanium or Zirconium crosslinkers will interact with cationic potato starch in flake form to cause the crosslinking to be substantially permanent. Thus, in this case, the “setting” of the cationic potato starch in the product may designed to be substantially permanent.

Cationic potato starch in flake form also has advantageous uses as an adhesive. An improvement over conventional adhesives in the manufacture of cigarettes is but one example. In service such as this, the adhesive needs to be a highly effective adhesive when cured, and further inexpensive, safe and easy to use in mass manufacture, and advantageously non-synthetic. The excellent film forming, adhesive, curing and delivery properties of cationic potato starch in flake form will be seen to fulfill these needs handsomely.

Cationic potato starch also has advantageous uses in agriculture as a drift control agent. As described in more detail above in the summary section, use of cationic potato starch with sprayed pesticide brings several advantages. As a chemically inert and naturally-derived product, cationic potato starch is compatible with both plants and the pesticides that are used on them. The superior film forming and adhesive properties of this starch encourage the pesticide to distribute evenly on the plant and then adhere well. Less pesticide is needed to dust the crop effectively, and less pesticide is likely to run off the plant, either by over spray or precipitation. More pesticide is distributed evenly on the plant, and then stays there. Less pesticide ends up either in the soil or the irrigation.

Cationic potato starch also has advantageous uses as a flocculent or a coagulant. As suggested above in the summary section, this starch may be added to industrial materials such as drilling mud or wastewater. Cationic potato starch's strong covalent bonds will form strong attractions for anionic impurities in such industrial materials, facilitating the removal of such impurities. The abilities of this starch to disperse quickly and evenly within the industrial materials will further assist this starch in achieving superior performance.

The advantages of cationic potato starch in the new applications described above will be now illustrated with reference to the following example.

### EXAMPLE 1

Approximately 5.0 grams of cationic potato starch in flake form was mixed with approximately 500 ml of de-ionized water in an overhead mixer for 10 minutes. The starch was Emprasol N available from Emsland Starch Gmbh.

The viscosity of the solution was measured on a Brookfield RVT viscometer using spindle #2 at 20 rpm. The viscosity was at about 500 cps.

Various crosslinking agents were added, including borax, titanium, or zirconium crosslinker in solutions of 0.01-1.0%. Viscosity was again measured on a Brookfield RVT viscometer using spindle #2 at 20 rpm. The viscosity was now measured at about 10,000-50,000 cps.

Subjective testing of this solution as hair styling gel indicated it to be a superior in hold and feel. Used as a conditioner, it was determined subjectively to be advantageous and rinsed off easily.

The solution was then used as a flocculant in waste water. Again, subjective testing indicated superior flocculation performance to conventional flocculants.

Although the present invention and its advantages have been described in detail, it should be understood that various changes, substitutions and alterations can be made herein without departing from the spirit and scope of the invention as defined by the appended claims.

I claim:

1. A personal care product comprising:
a potato starch in solid flake form having a substitution value of about at least 0.01;
the potato starch having been chemically modified to be cationic.

2. The personal care product of claim 1, in which the potato starch, originally in solid flake form, is converted to powder form prior introduction into said personal care product.

3. The personal care product of claim 1, in which the potato starch, originally in solid flake form, is converted to liquid form prior introduction into said personal care product.

4. The personal care product of claim 1, further comprising:
a crosslinking agent disposed to encourage crosslinking of the potato starch.

5. An adhesive comprising:
a potato starch in solid flake form having a substitution value of about at least 0.01;
the potato starch having been chemically modified to be cationic.

6. The adhesive of claim 5, in which the potato starch, originally in solid flake form, is converted to powder form prior introduction into said adhesive.

7. The adhesive of claim 5, in which the potato starch, originally in solid flake form, is converted to liquid form prior introduction into said adhesive.

8. A agricultural drift control agent comprising:
a potato starch in solid flake form having a substitution value of about at least 0.01;
the potato starch having been chemically modified to be cationic.

9. The agricultural drift control agent of claim 8, in which the potato starch, originally in solid flake form, is converted to powder form prior introduction into said agricultural drift control agent.

10. The agricultural drift control agent of claim 8, in which the potato starch, originally in solid flake form, is converted to liquid form prior introduction into said agricultural drift control agent.

11. A flocculent comprising:
a potato starch in solid flake form having a substitution value of about at least 0.01;
the potato starch having been chemically modified to be cationic.

12. The flocculent of claim 11, in which the potato starch, originally in solid flake form, is converted to powder form prior introduction into said flocculent.

13. The flocculent of claim 11, in which the potato starch, originally in solid flake form, is converted to liquid form prior introduction into said flocculent.

14. A coagulant comprising:
a potato starch in solid flake form having a substitution value of about at least 0.01;
the potato starch having been chemically modified to be cationic.

15. The coagulant of claim 14, in which the potato starch, originally in solid flake form, is converted to powder form prior introduction into said coagulant.

16. The coagulant of claim 14, in which the potato starch, originally in solid flake form, is converted to liquid form prior introduction into said coagulant.
